(19) United States
(12) Patent Application Publication
(10) Pub. No.: US 2002/0120182 A1
(43) Pub. Date: Aug. 29, 2002

(54) Endoscopic catheter

(75) Inventors: Dirk Muessig, Nuernberg (DE); Max Schaldach, Erlangen (DE); Max Schaldach JR., Berlin (DE)

Correspondence Address: HAHN LOESER & PARKS, LLP, TWIN OAKS ESTATE, 1225 W. MARKET STREET, AKRON, OH, 44313, US

(73) Assignee: Biotronik Mess-und Therapiegeraete GmbH & Co. Ingeniuerbaero Berlin

(21) Appl. No.: 10/072,698

(22) Filed: Feb. 8, 2002

(30) Foreign Application Priority Data
Feb. 10, 2001 (DE) 101 07 586.3

(51) Int. Cl. A61B 1/06

(52) U.S. Cl. 600/178; 600/104; 600/116

(57) ABSTRACT

An endoscopic catheter adapted for insertion into body cavities, comprising a distal catheter portion and an illumination device (40) which is adapted to illuminate an area around the distal catheter portion with electromagnetic radiation, and an image recording unit (60) which is adapted to record an image of the electromagnetic radiation reflected by the area around the distal catheter portion and pass it to the proximal end of the catheter (10), and an image reproduction unit (62) which is connected to the proximal end of the catheter (10) and adapted to reproduce an image of the recorded electromagnetic radiation, characterized in that the catheter (10) is adapted controllably for insertion into blood vessels, in particular blood vessels, and adapted to reproduce an image of the electromagnetic radiation reflected by the area around the distal catheter portion, with a wavelength for which blood has a high transparency.

The invention relates to a catheter for insertion into body cavities. The catheter includes a distal catheter portion, an illumination device, an image recording unit and an image reproduction unit. The illumination device is adapted to illuminate an area around the distal catheter portion with electromagnetic radiation. The image recording unit is adapted to record an image of the electromagnetic radiation reflected by the area around the distal catheter portion and pass it to the distal end of the catheter. The image reproduction unit is connected to the proximal end of the catheter and adapted to reproduce an image of the recorded electromagnetic radiation.

### BACKGROUND OF THE ART

Endoscopic catheters have long been known, for example, from U.S. Pat. No. 6,110,106 to MacKinnon, and serve for the visual investigation of body cavities.

A problem arises however if the body cavities to be investigated are filled, thus for example blood vessels are filled with blood.

### SUMMARY OF THE INVENTION

According to the invention, that problem is resolved in an endoscopic catheter of the kind set forth in the opening part of this specification, which is adapted controllably for insertion in body vessels, in particular blood vessels, and adapted to reproduce an image of the electromagnetic radiation reflected by the area around the distal catheter portion, with a wavelength for which the blood has a high transparency. A catheter of that kind advantageously permits even blood-filled vessels to be endoscopically investigated. That makes it possible in particular to control the catheter optically to a specific location at which for example a vessel dilation operation is to be implemented, a stent is to be fitted or body tissue is to be electrically stimulated.

Preferably the catheter is adapted to reproduce an image in a wavelength range of between 600 and 650 nanometers. For that purpose in particular the illumination device is adapted to illuminate the area around the distal catheter portion with light of a wavelength of between 600 and 650 nanometers. For that purpose the illumination device is preferably equipped with a band pass filter for a frequency band of between 600 and 650 nanometers. In addition the illumination device preferably includes a light waveguide from the proximal catheter end to the distal catheter end, which is adapted to pass electromagnetic radiation serving for illumination purposes, from the proximal catheter end to the distal catheter end. If the band pass filter is advantageously arranged at the proximal catheter end, only the electromagnetic radiation required for illumination purposes needs to be passed through the light waveguide, and the light waveguide can be optimally adapted to the wavelength thereof.

Preferably the endoscopic catheter is in the form of an electrode line and for that purpose is provided at its distal catheter portion with at least one electrode for delivering and/or picking up electrical signals to or from body tissue adjoining the distal catheter portion. A catheter of that kind, which for example is provided with stimulation electrodes, can advantageously be optically controlled to move to its target location.

In an alternative advantageous embodiment the distal catheter portion is provided with an expandable balloon. Preferably the expandable balloon is either suitable for dilation of constricted body vessels or it is adapted for the insertion and expansion of stents. When using a catheter of that kind, it is possible to expand a vessel under optical control or to set and expand a stent. In the last-mentioned variant the set and expanded stent can be easily optically monitored.

Preferably the catheter is provided with per se known control means for targeted deflection of the distal end of the catheter. That permits the catheter to be controlled in per se known manner and at the same time affords the advantage of endoscopic monitoring of catheter control.

### BRIEF DESCRIPTION OF THE INVENTION

The invention will now be described in greater detail by means of an embodiment with reference to the drawings in which identical parts are identified by identical reference numbers and in which:

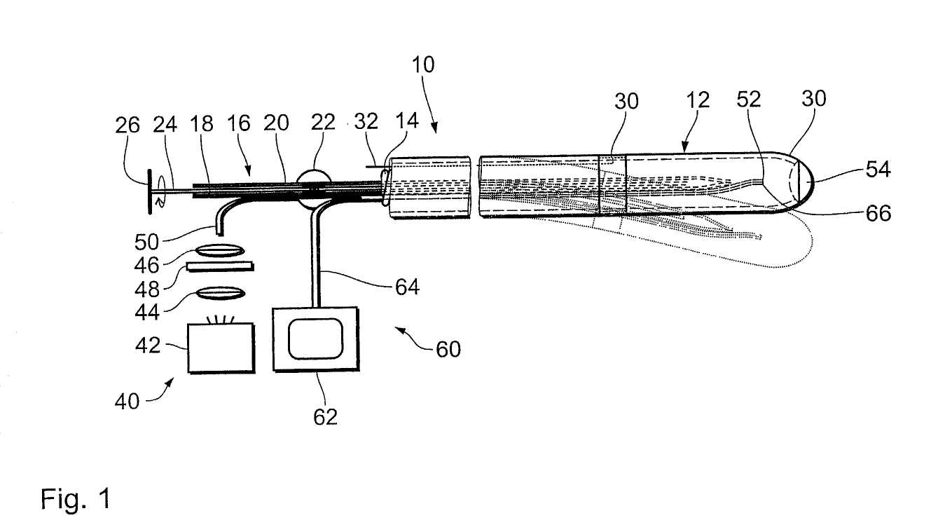

FIG. 1 shows an overview of an endoscopic catheter according to the invention,

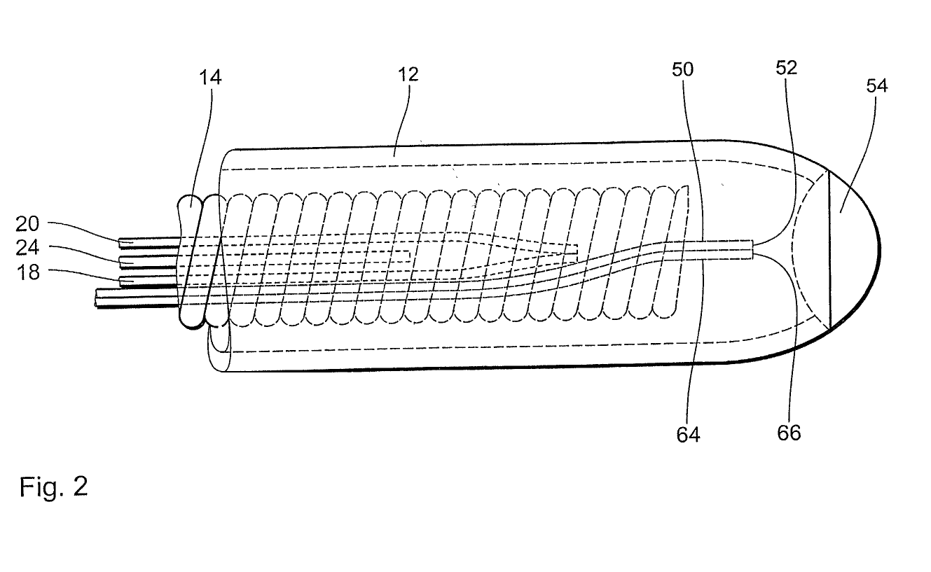

FIG. 2 shows the distal end of the endoscopic catheter of FIG. 1 on an enlarged scale,

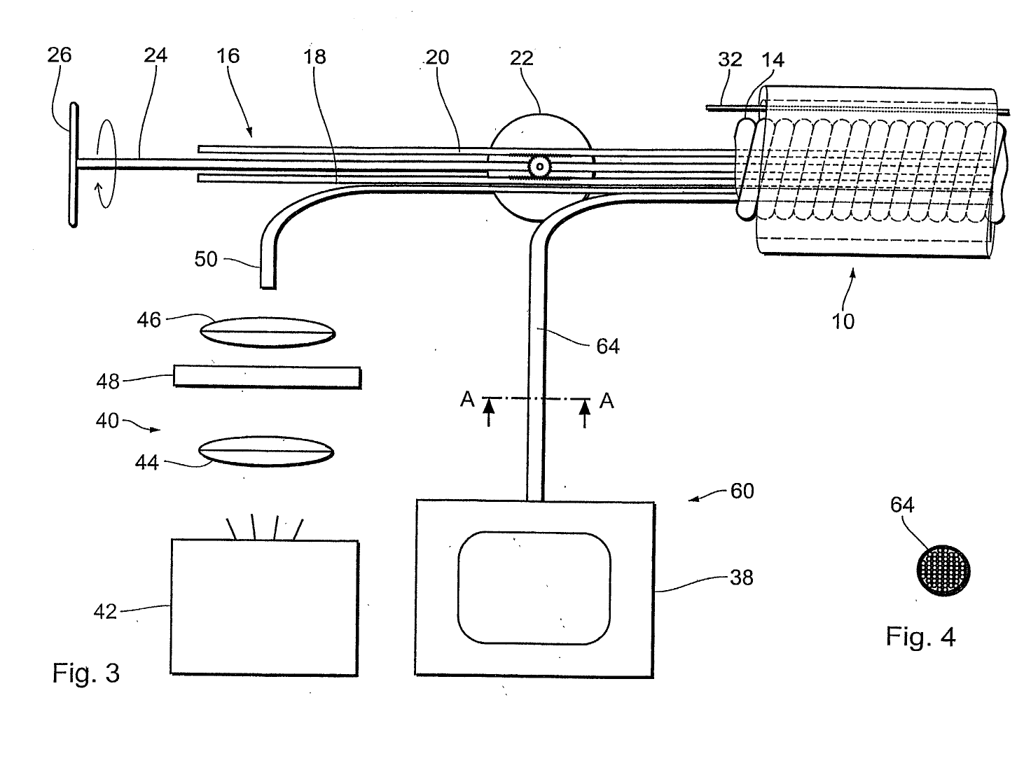

FIG. 3 shows the proximal end of the endoscopic catheter with an illumination and image reproduction unit connected thereto, and

FIG. 4 shows by way of example a view in cross-section through an optical fiber as is illustrated in FIG. 3.

### DETAILED DESCRIPTION OF THE INVENTION

The endoscopic catheter 10 shown in FIG. 1 essentially includes three functional groups: the first functional group includes a controllable catheter which is suitable for insertion into blood vessels, with a sheath 12 which encloses a stiffening flexible helical coil 14 which in its interior has a lumen for receiving control means 16 which include two control wires 18 and 20 which are connected together at their distal end and which in the region of their proximal end are displaceable lengthwise relative to each other by way of a hand wheel 22. In the region of its distal end, the catheter 10 is more flexible than in the regions adjoining same, as far as the proximal end. The distal end of the catheter can be specifically laterally deflected or diverted, as shown in broken line in FIG. 1, by rotation at the hand wheel 22 and thus by longitudinal displacement of the two control wires 18 and 20 relative to each other.

Provided between the two control wires 18 and 20 is a substantially torsionally stiff flat band 24 which, at least in the region of the distal end of the catheter 10, can be laterally deflected in the direction of the two control wires 18 and 20. The flat band 24 is connected to a handle portion 26 with which the flat band 24 can be rotated about its longitudinal axis. With rotation of the flat band 24, at least the two control wires 18 and 20 are also rotated so that the radial direction of a deflection movement of the catheter end by means of the two control wires 18 and 20 can be determined by means of the handle portion 26 and the flat band 24.

In the case of the catheter shown in FIG. 1, a further functional unit is formed by a stimulation or sensing electrode 30 in the region of the distal end of the catheter 10, which is electrically connected by way of an electric line 32 to the proximal end of the catheter 10. Instead of the one electrode 30, it is possible to provide a plurality of electrodes and a corresponding plurality of electric lines. A catheter of that kind can be used as a stimulation electrode line for example in connection with a cardiac pacemaker or a defibrillator.

In an alternative configuration (not shown), the catheter 10 can also be a balloon catheter which at its distal end carries an expandable balloon which for example is suitably adapted for enlarging vessels or for inserting stents into constricted blood vessels.

A third functional group serves for endoscope-like image recording of the area around the distal end of the catheter 10. This third functional group includes an illumination unit 40 with a light source 42, a lens system 44 and 46, an optical band pass filter 48 and an illumination light waveguide 50. In this case the illumination unit 40 is arranged in the region of the proximal end of the catheter 10 and the illumination light waveguide 50 extends from the proximal end of the catheter 10 into the proximity of the catheter tip at the distal end of the catheter 10. The illumination unit 40 is so designed that electromagnetic radiation serving for illumination purposes, in the wavelength range of between 600 and 650 nanometers, is passed to the distal end of the catheter 10 and can issue there in order to illuminate the area around the catheter tip. In that case, the radiation in the wavelength range of between 600 and 650 nanometers issues at a distal end 52 of the illumination light waveguide 50 and passes through an optical lens 54 at the distal end of the catheter 10. The lens 54 and the relative position of the distal illumination light waveguide 52 with respect to the focus of the lens 54 are so selected that the infra-red light issuing from the illumination light waveguide 50 is so distributed that the area around the distal end of the catheter 10 is uniformly illuminated. The wavelength of the electromagnetic radiation or the infra-red light in the range of between 600 and 650 nanometers is so selected that it is in a range in which blood is substantially transparent. In that way the illuminating radiation can pass through blood which is present in the blood vessels and is only reflected by the walls of the blood vessels.

The third functional unit also includes an endoscopic image recording and reproduction unit 60 which includes an image reproduction device 62 which is connected by way of an image light waveguide 64 to the distal end of the catheter 10. The image light waveguide 64 terminates at the focus of the lens 54 and is so designed that it can record an image of the area around the distal catheter end, which is projected onto a distal end face 66 of the image light waveguide 64, and can reproduce it at its proximal end. For that purpose the proximal end of the image light waveguide 64 is connected to the image reproduction unit 62. This includes a CCD-chip, onto which is projected an image of the area around the distal catheter end, which is transmitted optically to the proximal end of the image light waveguide 64. That image is an infra-red image in the wavelength range of between 600 and 650 nanometers and is electronically converted into a visible image which is represented on a display screen of the image reproduction unit 62.

FIG. 4 shows a view in cross-section through the image light waveguide 64 which includes many individual optical fibers of which only a few are shown in FIG. 4, for the sake of clarity of the drawing. Each individual fiber transmits a pixel of the recorded image.

In that way, a doctor when introducing the catheter 10, can continuously observe on the display screen of the image reproduction unit 62 the image, which is recorded by the distal catheter tip, of the vessel walls between which the tip of the catheter 10 is just being guided. That advantageously enables the doctor to detect vessel branchings into which the distal catheter tip is to be moved. The doctor can also see whether the catheter tip is already in the region of a vessel constriction to be treated, which is to be treated with a balloon integrated into the distal end of the catheter. If the distal end of the catheter is in the form of an electrode line, as illustrated in the Figures, the electrode line can be accurately positioned at the intended locations, with optical monitoring.

In a development the distal catheter end can include a plurality of optical systems and image light waveguides, with which images can be recorded not only by way of the catheter tip but also at locations of the catheter which are somewhat remote from the catheter tip, for example in the region of the electrode 30.

Likewise, it is possible to use alternative illumination and image reproduction units, just as the use of alternative control means is a possibility.

What is claimed is:

1. An endoscopic catheter adapted for insertion into body cavities, comprising:
a distal catheter portion;
an illumination device for illluminating an area around the distal catheter portion with electromagnetic radiation;
an image recording unit for recording an image of the electromagnetic radiation reflected by the area around the distal catheter portion and pass it to a proximal end of the catheter;
an image reproduction unit, connected to the proximal end of the catheter and adapted to reproduce an image of the recorded electromagnetic radiation,
wherein the catheter is adapted controllably for insertion into blood vessels, in particular blood vessels, and for reproducing the electromagnetic radiation image reflected by the area around the distal catheter portion, with a wavelength for which blood has a high transparency.

2. The catheter of claim 1, wherein the catheter reproduces an image recorded in a wavelength range of between 600 and 650 nanometers.

3. The catheter of claim 2, wherein the illumination device illuminates the area around the distal catheter portion with infra-red light of a wavelength of between 600 and 650 nanometers.

4. The catheter of claim 3, wherein the illumination device further comprises an optical band pass filter for a frequency band of between 600 and 650 nanometers.

5. The catheter of claim 4, wherein the illumination device further comprises an illumination light waveguide from the proximal to a distal catheter end, to pass electromagnetic radiation serving for illumination purposes from the proximal catheter end to the distal catheter end.

6. The catheter of claim 1, wherein the illumination device further comprises an illumination light waveguide from the proximal to a distal catheter end, to pass electromagnetic radiation serving for illumination purposes from the proximal catheter end to the distal catheter end.

7. The catheter of claim 2, wherein the illumination device further comprises an illumination light waveguide from the proximal to a distal catheter end, to pass electromagnetic radiation serving for illumination purposes from the proximal catheter end to the distal catheter end.

8. The catheter of claim 3, wherein the illumination device further comprises an illumination light waveguide from the proximal to a distal catheter end, to pass electromagnetic radiation serving for illumination purposes from the proximal catheter end to the distal catheter end.

9. The catheter of claim 5, wherein the catheter is in the form of an electrode line and for that purpose is provided at its distal catheter portion with at least one electrode for delivering and/or picking up electrical signals to or from body tissue adjoining the distal catheter portion.

10. The catheter of claim 1, wherein the catheter is in the form of an electrode line and for that purpose is provided at its distal catheter portion with at least one electrode for delivering and/or picking up electrical signals to or from body tissue adjoining the distal catheter portion.

11. The catheter of claim 2, wherein the catheter is in the form of an electrode line and for that purpose is provided at its distal catheter portion with at least one electrode for delivering and/or picking up electrical signals to or from body tissue adjoining the distal catheter portion.

12. The catheter of claim 3, wherein the catheter is in the form of an electrode line and for that purpose is provided at its distal catheter portion with at least one electrode for delivering and/or picking up electrical signals to or from body tissue adjoining the distal catheter portion.

13. The catheter of claim 4, wherein the catheter is in the form of an electrode line and for that purpose is provided at its distal catheter portion with at least one electrode for delivering and/or picking up electrical signals to or from body tissue adjoining the distal catheter portion.

14. The catheter of claim 6, wherein the catheter is in the form of an electrode line and for that purpose is provided at its distal catheter portion with at least one electrode for delivering and/or picking up electrical signals to or from body tissue adjoining the distal catheter portion.

15. The catheter of claim 7, wherein the catheter is in the form of an electrode line and for that purpose is provided at its distal catheter portion with at least one electrode for delivering and/or picking up electrical signals to or from body tissue adjoining the distal catheter portion.

16. The catheter of claim 8, wherein the catheter is in the form of an electrode line and for that purpose is provided at its distal catheter portion with at least one electrode for delivering and/or picking up electrical signals to or from body tissue adjoining the distal catheter portion.

17. The catheter of claim 9, wherein the catheter carries an expandable balloon at its distal catheter portion.

18. The catheter of claim 1, wherein the catheter carries an expandable balloon at its distal catheter portion.

19. The catheter of claim 2, wherein the catheter carries an expandable balloon at its distal catheter portion.

20. The catheter of claim 3, wherein the catheter carries an expandable balloon at its distal catheter portion.

21. The catheter of claim 4, wherein the catheter carries an expandable balloon at its distal catheter portion.

22. The catheter of claim 14, wherein the catheter carries an expandable balloon at its distal catheter portion.

23. The catheter of claim 15, wherein the catheter carries an expandable balloon at its distal catheter portion.

24. The catheter of claim 16, wherein the catheter carries an expandable balloon at its distal catheter portion.

25. The catheter of claim 17, wherein the expandable balloon is suitably adapted for dilation of constricted blood vessels.

26. The catheter of claim 18, wherein the expandable balloon is suitably adapted for dilation of constricted blood vessels.

27. The catheter of claim 19, wherein the expandable balloon is suitably adapted for dilation of constricted blood vessels.

28. The catheter of claim 20, wherein the expandable balloon is suitably adapted for dilation of constricted blood vessels.

29. The catheter of claim 21, wherein the expandable balloon is suitably adapted for dilation of constricted blood vessels.

30. The catheter of claim 22, wherein the expandable balloon is suitably adapted for dilation of constricted blood vessels.

31. The catheter of claim 23, wherein the expandable balloon is suitably adapted for dilation of constricted blood vessels.

32. The catheter of claim 24, wherein the expandable balloon is suitably adapted for dilation of constricted blood vessels.

33. The catheter of claim 25, wherein the balloon is suitably adapted for inserting and expanding stents.

34. The catheter of claim 26, wherein the balloon is suitably adapted for inserting and expanding stents.

35. The catheter of claim 27, wherein the balloon is suitably adapted for inserting and expanding stents.

36. The catheter of claim 28, wherein the balloon is suitably adapted for inserting and expanding stents.

37. The catheter of claim 29, wherein the balloon is suitably adapted for inserting and expanding stents.

38. The catheter of claim 30, wherein the balloon is suitably adapted for inserting and expanding stents.

39. The catheter of claim 31, wherein the balloon is suitably adapted for inserting and expanding stents.

40. The catheter of claim 32, wherein the balloon is suitably adapted for inserting and expanding stents.

41. The catheter of claim 33, wherein the catheter further comprises means for controlling a targeted deflection of the distal end of the catheter, actuable from the proximal end thereof.

42. The catheter of claim 1, wherein the catheter further comprises means for controlling a targeted deflection of the distal end of the catheter, actuable from the proximal end thereof.

43. The catheter of claim 2, wherein the catheter further comprises means for controlling a targeted deflection of the distal end of the catheter, actuable from the proximal end thereof.

44. The catheter of claim 3, wherein the catheter further comprises means for controlling a targeted deflection of the distal end of the catheter, actuable from the proximal end thereof.

45. The catheter of claim 4, wherein the catheter further comprises means for controlling a targeted deflection of the distal end of the catheter, actuable from the proximal end thereof.

46. The catheter of claim 5, wherein the catheter further comprises means for controlling a targeted deflection of the distal end of the catheter, actuable from the proximal end thereof.
